(19) United States
(12) Patent Application Publication
(10) Pub. No.: US 2009/0233941 A1
(43) Pub. Date: Sep. 17, 2009

(54) Co-Crystals of Calixarenes and Biologically Active Molecules

(75) Inventors: Anthony William Coleman, Caluire (FR); Adina Nicoleta Lazar, Villeurbanne (FR); Kinga Suwinska, Warszaw (PL); Oksana Danylyuk, Mostys'Ka district (UA)

Correspondence Address: ARENT FOX LLP, 1050 CONNECTICUT AVENUE, N.W., SUITE 400, WASHINGTON, DC, 20036, US

(73) Assignees: Centre National de la Recherche Scientifique-CNRS, Paris Cedex 16 (FR); Universie' Claude Bernard de Lyon 1, Villeurbanne Cedex (FR); Institute of Physical Chemistry-Polish Academy of Sciences, Warsaw (PL)

(21) Appl. No.: 12/226,418

(22) PCT Filed: Apr. 18, 2007
(86) PCT No.: PCT/FR2007/051129

§ 371 Date: May 21, 2009

(30) Foreign Application Priority Data
Apr. 18, 2006 (FR) 0603405

(51) Int. Cl. A61K 31/506; A61K 31/155; A61K 31/245; A61K 31/138; A61K 31/4178; C07C 309/43

(52) U.S. Cl. 514/252.14; 514/635; 514/536; 514/651; 514/397; 562/80

(57) ABSTRACT

This invention relates to co-crystals of at least one molecule of calyx[n]arene or at least one of its derivatives, and at least one biologically active molecule, compositions and medicines including them, and the use of these co-crystals and methods used to obtain them.

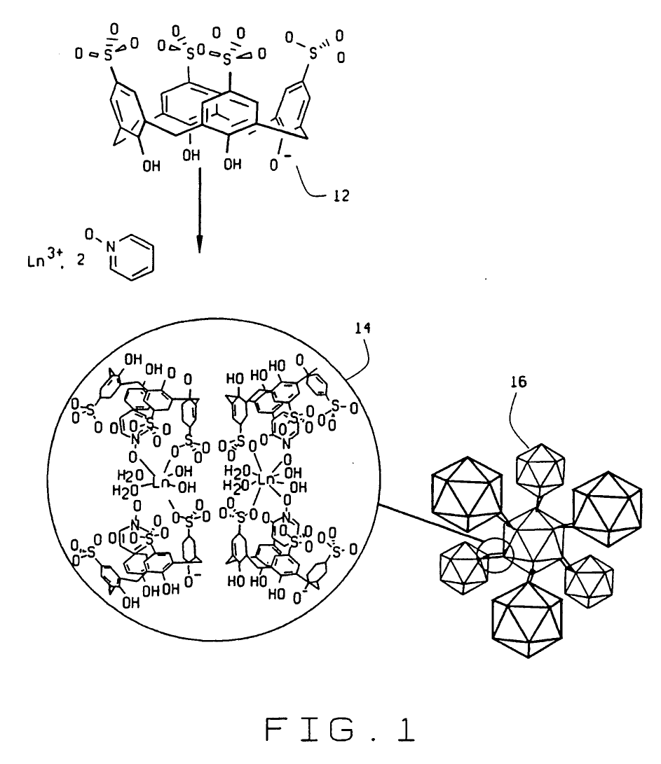

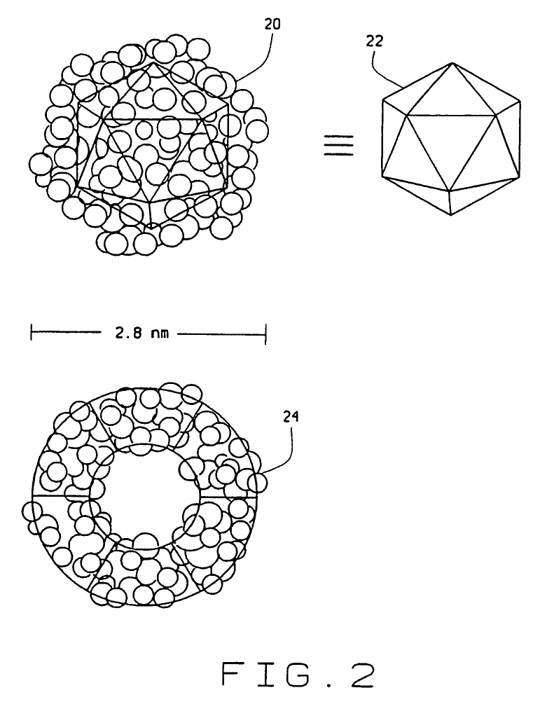

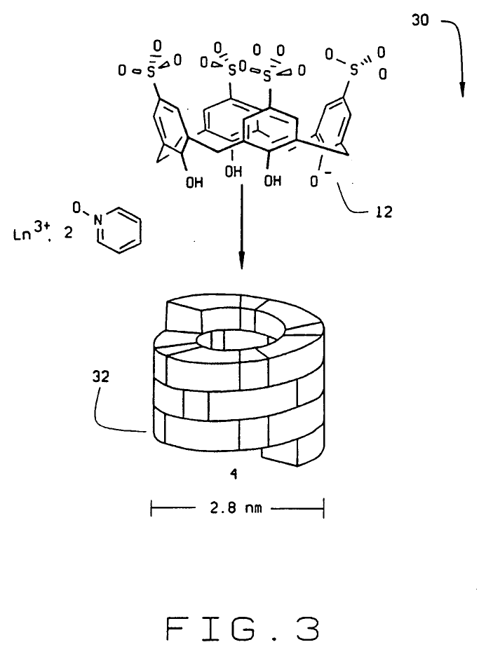

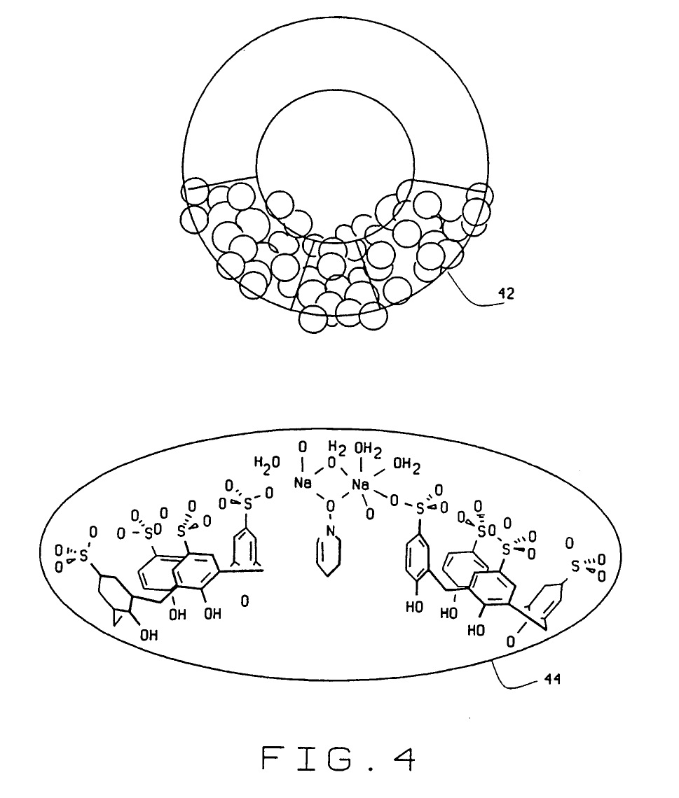

The present international application claims priority right over the French patent application N° 06/03405 filed on 18 Apr. 2006. The content of this priority document is hereby incorporated by reference in its entirety.

The present invention concerns the field of organic co-crystals. More precisely, this invention concerns derivatives of calixarenes and of similar macrocyclic compounds for the formation of co-crystals and pharmaceutical compositions comprising these co-crystals. The invention also concerns methods of preparing these novel co-crystals and their use, in particular in the preparation of medications.

Biologically active compounds, in particular pharmaceutically active compounds (PACs), can be prepared in various forms. They can be in amorphous form or in crystalline form, in particular in medications.

The pharmaceutical industry often favours compounds in crystalline form since a simple crystallisation step makes it possible to isolate and purify them.

Among the crystals of biologically active compounds, some may have problems in terms of solubility, stability, hygroscopy, biocompatibility and/or polymorphism.

There therefore remains a need for biologically active compounds in crystalline form having improved properties.

Thus the inventors discovered that specific co-crystals made it possible to resolve the problems mentioned above in whole or in part.

According to a first aspect, an object of the invention is a co-crystal of at least one calix[n]arene molecule or at least one of its derivatives, and at least one biologically active molecule.

Naturally this co-crystal has the physical properties corresponding to crystalline compounds, for example a diffraction of x-rays due to an ordered organisation in three dimensions.

The co-crystal of calix[n]arene, or one of its derivatives, and a biologically active molecule, in particular a pharmaceutically active compound (PAC), can be defined as the association of at least one calix[n]arene molecule or one of its derivatives and at least one biologically active molecule.

This association can in particular take place by means of at least one non-covalent interaction. When several non-covalent interactions are present, it may be a case of the same type of non-covalent interaction or of several types of non-covalent interaction.

Among non-covalent interactions, ionic, ion-dipole, dipole-dipole, induced dipole-dipole, induced dipole-induced dipole, hydrogen bond, π-π interaction, Van der Waals force and hydrophobic interaction can be cited.

According to a particular embodiment, the calix[n]arene or its derivative, and the biologically active molecule, are not linked by one or more covalent bonds.

Calix[n]arenes and their derivatives, in particular resorcinarenes, are host macrocycles of molecules. They may be available in industrial quantities and in pharmaceutical quality purity.

These compounds can generally be modified both with regard to the aromatic cycles and the benzylic positions or on the phenol functions. Such modifications can make it possible to modulate, and in particular to increase, the interactions with various groups present on host molecules, in particular on biologically active molecules, and in particular on molecules present as an active principle in medications.

Calix[n]arenes are cyclic compounds comprising several aromatic groups.

In particular, calix[n]arene may comply with the following formula (I):

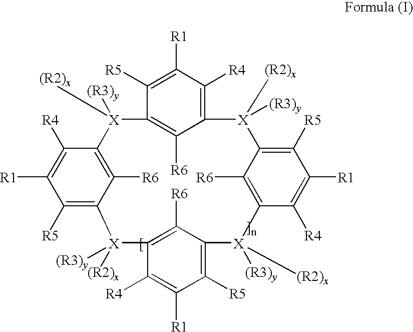

in which:
n is an integer number chosen from 1, 2, 3, 4, 5, 6, 7, 8, 9, 10, 11, 12, 13, 14, 15, 16, 17, 18, 19, and 20,
R1 represents a polar group, in particular chosen from the group comprising the hydroxyl, ether, carboxylic acid, sulfonic acid, phosphonic acid, sulfonamide, amide and ester functions, or an alkyl, alkene, alkyne or acyl group, linear, branched or cyclic, or an aryl, arylalkyl or alkylaryl group, or heterocyclic compound, possibly substituted, in particular by a polar group,
R2, R3, R4 and R5 each represent independently from one another a hydrogen atom, an alkyl, alkene or alkyne group, possible substituted, in particular by a polar group, in particular by a hydroxyl or ether function, in particular carrying an alkyl, alkene or alkyne group,
X represents an atom chosen from carbon, oxygen, sulfur and nitrogen,
x and y each represent independently of each other 0 or 1, and
R6 represents a polar group, in particular chosen from the group comprising the hydroxyl, ether, carboxylic acid, sulfonic acid, phosphonic acid, sulfonamide, amide and ester functions, or an alkyl group, branched or linear, possibly substituted by a polar group.

The alkyl, alkene or alkyne radicals may comprise 1 or 2 to 18 carbon atoms, in particular 1 or 2 to 12 carbon atoms, and especially 1 or 2 to 6 carbon atoms.

The alkene radicals may comprise one or more double bonds.

The alkyne radicals may comprises one or more triple bonds.

The aryl, arylalkyl or alkylaryl radicals may comprise 5 to 20 carbon atoms, in particular 6 to 15 carbon atoms.

The heterocyclic compounds may comprise 4 to 12 carbon atoms and at least one heteratom, in particular chosen from oxygen, sulfur and nitrogen.

“Polar group” means, within the meaning of the present invention, a group whose dipolar moment is different from zero. Among polar groups, the functions comprising at least one heteroatom, for example the hydroxyl, amine (primary, secondary and tertiary), ether, carboxylic acid, hydroxysulfate, sulfonic acid, hydroxyphosphate, phosphonic acid, sulfonamide, amine and ester functions can be cited.

In particular, the R4 and R5 groups are identical, and in particular represent a hydrogen atom.

According to one aspect of the invention, X is a carbon atom, and the carbon atoms carrying the R2 and R3 groups may all have the same configuration, and in particular all be (S) or all (R).

The said at least one calix[n]arene or the said at least one of its derivatives can be chosen from the group comprising calix[n]arene, (25,27-bis(dihydroxyphosphoriloxy)-26,28-dihydroxycalix[4]arene) dihydrophosphonic acid, calix[4]arene (5,11,17,23-tetrakis(p-sulfonic acid)-25,26,27,28-tetrahydroxycalix[4]arene)parasulfonic acid, dimethoxycarboxy calix[4]arene (25,27-bis(methoxycarboxy)-26,28 dihydroxycalix[4]arene, tetramethoxycarboxy calix[4]arene (25,26,27,28-tetra(methoxycarboxy)calyx[4]arene, tetraproprioxycarboxy calix[4]arene (25,26,27,28-tetra(proprioxycarboxy)calix[4]arene, calix[4]arene (5,11,17,23,29,35-hexasulfonic acid 37,38,39,40,41,42-hexahydroxycalix[6]arene parasulfonic acid, calix[8]arene (5,11,17,23,29,35,41,47-octasulfonic acid-50,51,52,53,54,56,57-octahydroxycalix[8]arene) parasulfonic acid, tetraacylcalix[4]arene(5,11,17,23-tetraacylcalix[4]arene), tetrahydroxycalix[4]arene), tetraacylcalix[6]arene(5,11,17,23,29,35-tetracylcalix[6]arene).

“Biologically active molecule” means, within the meaning of the present invention, a molecule having an activity vis-à-vis biological processes, in particular a pharmaceutically active principle, especially a molecule used or known as an active principle in a medication.

The said at least one biologically active molecule may comprise at least one unit able to form at least one non-covalent interaction with at least complementary unit of the said at least one calix[n]arene or of the said at least one of its derivatives.

The said at least one biologically active molecule may comprise at least one aromatic ring, an amine function, alkyl chains of greater or lesser length, etc, so as to form an interaction with the calixarene.

Among the biologically active molecules, the molecules used or known as a pharmaceutically active principle in at least one medication can be cited. This biologically active molecule may be chosen from the group comprising N,N′-bis(4-chlorophenyl)-3,12-diimino-2,4,11,13-tetraazatetra-decanediimidamide (Chlorhexidine®), 2-(dimethylamino)ethyl 4-(butylamino)benzoate (Tetracaine®), (Z)-2-[4-(1,2-diphenyl-1-butenyl)phenoxy]-N,N-dimethylethanamine (Tamoxifen®), (3S-cis)-3-ethyldihydro-4-[(1-methyl-1H-imidazol-5-yl)methyl]-2(3H)-furanone (Pilocarpine®) and 2-[4(1,3-Benzodioxol-5-ylmethyl)-1-piperazynyl]pyrimidine (Piribedil®).

The co-crystal according to the invention can have solubility in water, in mol·.l⁻¹, at 25° C. greater than or equal to 10%, in particular 20%, or even 30% with respect to the solubility of the biologically active molecule, in particular in neutral form and/or in the form of a salt, in particular potassium or ammonium salt. In particular vis-à-vis the form of the biologically active molecule, neutral or pharmaceutically acceptable salt, having the best solubilisation in water, or even in physiological fluids.

The co-crystal according the invention may have stability greater than 30%, or even 40%, with respect to the stability of the biologically active molecule, in particular in neutral form and/or in the form of a salt, in particular potassium or ammonium salt.

Moreover, the co-crystals according to the invention can also allow improvement of other properties of the biologically active molecules, such as an improvement of the passage of biological barriers, such as the membrane barrier, in particular of cells.

According to one of its aspects, an object of the invention is a pharmaceutical composition comprising at least one co-crystal of least one calix[n]arene, or of at least one or its derivatives, and at least one biologically active molecule in a pharmaceutically or physiologically acceptable carrier.

According to one aspect of the invention, in the composition, the said at least one calix[n]arene complies with formula (I), in which the variables R1, R2, R3, R4, R5, R6, X, n, x and y are as defined previously in this document.

The co-crystal may be present in the composition in a proportion ranging from 0.01 to 100% by weight, in particular 0.01 to 50% by weight, especially 1 to 25% by weight to with respect of the total weight of the composition.

This composition comprises at least one pharmaceutically acceptable carrier, in particular chosen from the group comprising lactose, starch, possible modified, cellulose, hydroxypropyl cellulose, hydroxypropylmethyl cellulose, mannitol, sorbitol, xylitol, dextrose, calcium sulphate, calcium phosphate, calcium lactate, dextrates, inositol, calcium carbonate, glycine, bentonite, polyvinylpyrriolidone and mixtures thereof.

The composition may comprise a pharmaceutically or physiologically acceptable carrier content ranging from 5% to 99.99% by weight, in particular 10% to 90% by weight, and especially 20% to 75% by weight with respect to the total weight of the composition.

The composition can also comprise at least one pharmaceutically or physiologically acceptable binder and/or adhesive. They may be chosen from the group comprising sucrose, gelatine, glucose, starches, alginic acid, aluminium and magnesium silicate, celluloses, PEGs, guar gum, polysaccharidic acids, bentonites, polymethacrylates, hydroxypropylcellulose and mixtures thereof.

The composition according to the invention can also comprise at least one pharmaceutically or physiologically acceptable lubricant. This can be chosen from the group comprising glyceryl beheptate, stearic acid and its salts, in particular magnesium, calcium and sodium, hydrogenated vegetable oils, colloidal silicas, talc, waxes, boric acid, sodium benzoate, sodium acetate, sodium fumarate, sodium chloride, DL-Leucine, PEGs, sodium oleate, sodium lauryl sulfate, and in particular magnesium stearate.

The composition may comprise a proportion of lubricant ranging from 0.1 to 10% by weight, and in particular 0.2 to 5% by weight with respect to the total weight of the composition.

The composition may also comprise other excipients, such as dyes, flavourings and sweeteners, in particular known in pharmacy.

According to yet another of its aspects, another object of the invention is the use of at least one calix[n]arene co-crystal, one of its derivatives and a biologically active molecule for the preparation of a medication in particular intended for treating infectious diseases, in particular bacterial and/or viral, parasitic diseases, fungal infection diseases, prion diseases, allergic illnesses, cardiovascular diseases, dermatological diseases, rare diseases (referred to as orphan diseases), genetic diseases, in particular classed by organ, apparatus or function or by region of the globe, chromosome diseases, in particular due to an abnormality in number or to a deletion, illnesses from intoxication, illnesses due to cell degenerescence, in particular cancers, leukaemias and Alzheimer's and Parkinson's diseases, environmental diseases, in particular obesity, alcoholism and hypertension, diseases caused by deficiencies, auto-immune and inflammatory diseases, diseases having an uncertain cause or multiple causes, diseases of the eye, and tissue and cell trauma.

According to one aspect of the invention, in the said medication, the said at least one calix[n]arene complies with formula (I) in which the variables R1, R2, R3, R4, R5, R6, X, n, x and y are as defined previously in this document.

The said medications according to the invention can be administered by different routes. By way of examples of administration routes that can be used for the medications according to the invention, the oral, rectal, cutaneous, pulmonary, nasal and sublingual routes, the parenteral route, in particular intradermic, subcutaneous, intramuscular, intravenous, intra-arterial, intra-rachidian, intra-articular, intrapleural and intraperitoneal routes can be cited.

The medications according to the invention can be administered on one or more occasions or in continuous release, in particular in continuous perfusion.

The medications according to the invention can be in various forms, in particular in a form chosen from the group comprising tables, capsules, pills, syrups, suspensions, solutions, powders, granules, emulsions, microspheres and injectable solutions, preferably tablets, injectable solutions, sublingual sprays and skin patches.

These various forms can be obtained by techniques well known to persons skilled in the art.

The formulations appropriate to administration by parenteral route, the pharmaceutically acceptable vehicles appropriate to this administration route and the corresponding formulation and administration techniques can be implemented according to methods well known to persons skilled in the art, in particular those described in the manual Remington's Pharmaceutical Sciences (Mack Publishing Co., Easton, Pa., 20^{th} edition, 200).

The co-crystal according to the invention can be present in the medication in a quantity ranging from 50 mg to 5 g per unit dose, in particular from 100 mg to 2 g.

The medication according to the invention can be administered in one or more doses per day, preferably in 1 to 4 doses per day.

According to another of its aspects, an object of the invention is a method of preparing co-crystals of at least one calix[n]arene, or at least one of its derivatives, with at least one biologically molecule comprising at least the steps consisting of:
in at least one solvent, putting at least one calix[n]arene, or at least one of its derivatives, together with at least one biologically active molecule, and
recovering said co-crystals.

According to one aspect of the invention, in the said preparation method, the said at least one calix[n]arene complies with formula (I) in which the variables R1, R2, R3, R4, R5, R6, X, n, x and y are as defined previously in this document.

More particularly, the preparation of the co-crystals according to the invention is carried out according to a crystallisation method with several solvents, in particular belonging to the same chemical class. Among the solvents able to be used, ethanol and water can be cited. The ethanol may make it possible to prepare a solution of a calix[n]arene or one of it derivatives, and the water to prepare a solution of a biologically active molecule.

The method of preparing co-crystals according to the invention may comprise at least the steps consisting of:
slowly adding an alcohol solution of calix[n]arene, or one its derivatives, to an aqueous solution of biologically active molecules so that crystallisation can take place, in particular at the interface of these two phases, and
recovering the co-crystals obtained.

More precisely, the method of preparing the co-crystals may comprise the following steps;
addition of an aqueous solution comprising a biologically active molecule,
addition of distilled water,
addition of ethanol,
addition of a solution of calix[n]arene or one of its derivatives,

so as to form an interface, in particular visible, between the two solvents,

and then recovering the crystals that form, in particular at the interface.

Among the crystallisation methods that can be used according to the invention, those described in the work “Crystallization of Nucleic Acids and Proteins—A Practical Approach” (A Ducruix and R Giege, Oxford University Press, 2^{nd} edition, 1999) can be cited.

The crystal formation time is in general around a few days.

According to one of its aspects, an object of the invention is the use of calix[n]arene or one of its derivatives for preparing a calix[n]arene/biologically active molecule co-crystal.

According to one aspect of the invention, the said calix[n]arene complies with formula (I) in which the variables R1, R2, R3, R4, R5, R6, X, n, x and y as defined previously in this document.

According to another of its aspects, another object of the invention is the use of calix[n]arene, or one of its derivatives, as an agent allowing the formation of a co-crystal with a biologically active molecule.

According to one aspect of the invention, the said calix[n]arene complies with formula (I) in which the variables R1, R2, R3, R4, R5, R6, X, n, x and y are as defined previously in this document.

The following examples are given by way of illustration and can under no circumstances serve to limit the invention.

### EXAMPLES

### I Protocols

### I.1 Crystal Growth

The procedure of preparing co-crystals is implemented by carefully pouring the following solutions into a tube and in the following order, so as to obtain an ethanol/water interface:
1 ml of a 0.01 M solution (S1) of calix[n]arene in ethanol,
1 ml of 95% ethanol,
1 ml of distilled water, and
1 ml of a 0.01 M solution (S2) of biologically active molecule in distilled water.

Then the tube is closed. The co-crystals form at the water/ethanol interface and are recovered after four days.

### I.2 Collection of Data

All the diffraction instruments have the following components: radiation source (KCCD diffractometer), a mount for the positioning of the sample, a detector and a computerised data checking and collection system.

A monocrystal is selected and mounted on the sample support, aligning the centre of gravity; fixing of the collection parameters (temperature in the chamber of the sample, distance of detector with respect to the sample, interval and degree of rotation of crystal).

The crystals have a periodic molecular arrangement by virtue of a stack of identical planes in a crystal. These structural properties allow analysis of the crystals by x-ray diffraction. The distances between each crystalline plane correspond to the reticular distance.

The characteristics of the crystal are measured and calculated according to Bragg's law:

2dₕₖₗ sin [symbol] ₕₖₗ=└ where
dₕₖₗ represents the reticular distance, the indices designating the relevant direction in the crystal,
└ represents the monochromatic radiation wavelength, and
2 [symbol] ₕₖₗ represents the angle between the incident ray and the diffracted ray.

Analysis of the collected data commences with the identification of the intensity peaks followed by the indexing of the diffraction spots. From this diffraction map, the information on the arrangement of the molecules in the crystal is determined and subsequently the basic structure of the complex-elementary mesh. A possible list of space groups is given, and then the one that gives the lowest error is chosen. Thus the composition of the elementary mesh and the nature and number of the molecules making it up are learned.

The data of the mesh and the unique reflections are imported into WinGX software. The structure is resolved, the majority of the time using the Shelix program, by Direct Methods or Patterson. The structure is then refined, determining, step by step, the correspondence of the atoms in the structure to the molecules making up the crystal.

This can be carried out using the Shelx97 program. The hydrogen atoms are the last to be identified since their intensity is lower than that of the other atoms. Graphics programs such as ORTEP are often used to confirm the correct choice of the atoms by evaluating the thermal factors for each atom.

The correctness of the refinement is given by the value of the factor R. The lower this value, the more accurate the structure determined. All this refinement work is done with “.ins” and “.res” files, the existence of “.hkl” files being essential.

Once the structure has been completely determined, a “.cif” file is created. This file is used for analysing the structure

### I.3 Analysis of Interactions in the Complex

The rest consists of determining the interactions between the component molecules of the elementary mesh from the “.cif” file. For this purpose, evaluation of all intra- and intermolecular bonds, such as the hydrogen bonds, the dipolar interactions or the aromatic interactions, is necessary.

The software and program used are DS ViewerPro software and the Mercury program.

At the very start, it is desirable to identify all the intramolecular interaction elements; secondly the search for intermolecular interactions in all the components of the elementary mesh is carried out. A wider view is often necessary for this study, which requires the production of molecular “packings”. Thus interactions of the dipole-dipole, hydrogen bond or aromatic interactions type are determined.

### II Examples

### Example 1

### Chlorhexidine/calix[4]arene parasulfonic acid co-crystal

Calix[4]arene parasulfonic acid (5,11,17,23-tetrakis(p-sulfonic acid)-25,26,27,28-tetrahydroxycalix[4]arene has the following formula:

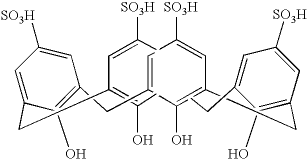

The chlorhexidine/calix[4]arene parasulfonic acid co-crystal is prepared according to the general procedure described above.

The crystallographic data are as follows:

| | Space | | | | | | | | | |
|---|---|---|---|---|---|---|---|---|---|---|
| Symmetry | group | A | B | C | α | β | γ | M | Volume | R |
| Triclinic | P-1 | 15.326 | 15.9820 | 16.1350 | 65.678 | 77.180 | 64.4270 | 543.47 | 32.4408 | 0.07 |
| | | (3) | (50 | (4) | (2) | (2) | (10) | | (17) | |

The direction interactions between the calix[4]arene parasulfonic acid and the chlorhexidine are:

| | Hydrogen bond | | | Dipolar interactions | | |
|---|---|---|---|---|---|---|
| | calixarene | medication | distance [λ] | calixarene | medication | distance [λ] |
| Functions | sulfonate | amine | 2.842; | aromatic | alkyl | 3.604; 3.812; |
| involved | | | 2.900; | cycle | | 3.876; 3.686; |
| | | | 2.816; | | | 3.882 |
| | | | 2.783; 3.283 | | | |
| | sulfonate | imine | 2.746 | sulfonate | imine | 3.419; 3.358 |
| | | | 2.841; 2.888 | | | |
| | | | | aromatic CH | Cl | 3.410; 3.508 |
| | | | | aromatic CH | aromatic CH | 3.757; 3.937 |

Conformations adopted by the chlorhexidine molecule in the co-crystal:

| | C4S-Chlorhexidine co-crystal | |
|---|---|---|
| Chlorhexidine | Conformation | Torsion angles |
| 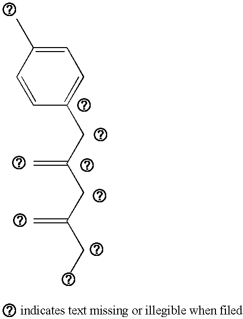 | 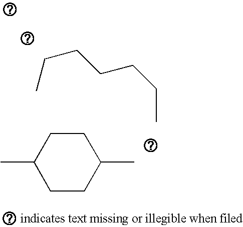 | C1 [symbol] N1 [symbol] C2 [symbol] N3 = −164.44 N1 [symbol] C2 [symbol] N3 [symbol] C3 = −147.99 C2 [symbol] N3 [symbol] C3 [symbol] N5 = 35.60 N3 [symbol] C3 [symbol] N5 [symbol] C4 = 174.88 C3 [symbol] N5 [symbol] C4 [symbol] C5 = 154.84 N5 [symbol] C4 [symbol] C5 [symbol] C6 = [symbol] C4 [symbol] C5 [symbol] C6 [symbol] C7 = [symbol] C5 [symbol] C6 [symbol] C7 [symbol] C8 = 52.85 C6 [symbol] C7 [symbol] C8 [symbol] C9 = 57.52 C7 [symbol] C8 [symbol] C9 [symbol] N6 = 179.33 C8 [symbol] C9 [symbol] N6 [symbol] C16 = 132.46 C9 [symbol] N6 [symbol] C10 [symbol] N8 = 174.70 N6 [symbol] C10 [symbol] N8 [symbol] C11 = −144.85 C10 [symbol] N8 [symbol] C11 [symbol] N10 = −151.66 N8 [symbol] C11 [symbol] N10 [symbol] C12 = 21.77 |
| 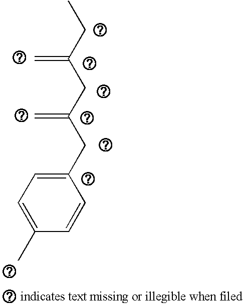 | | |

[symbol]

indicates data missing or illegible when filed

### Example 2

### Chorhexidine/calix[4]arene dihydrophosphonic acid

Calix[4]arene dihydrophosphonic acid (25,27-bis (dihydroxyphosphoriloxy)-26,28-dihydroxycalix[4]arene) is represented by the following formula:

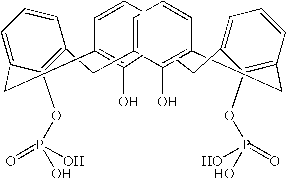

The chlorhexidine/calix[4]arene dihydrophosphonic acid co-crystal is prepared according to the general procedure described above.

The crystallographic data are:

| | Space | | | | | | | Molecular | | |
|---|---|---|---|---|---|---|---|---|---|---|
| Symmetry | group | A | B | C | α | β | γ | mass | Volume | R |
| monoclinical | Cc | 26.4440 | 33.1260 | 31.572 | 90.00 | 109.506 | 64.4270 | 1491.54 | 10329 | 0.07 |
| | | (13) | (16) | (3) | | (6) | (10) | | (17) | |

The direct interactions between the calix[4]arene dihydrophosphonic acid and chlorhexidine are:

| | Hydrogen bond | | | Dipolar interactions | | |
|---|---|---|---|---|---|---|
| | calixarene | medication | distance [λ] | calixarene | medication | distance [λ] |
| Functions | O | N amine | 2.911; 2.751; 3.099; | Aromatic | CH₂ | 3.630 |
| involved | phosphonate | | 2.784 | CH | | |
| | Sulfonate | imine | 2.746 | CH₂ | CH₂ | 3.751 |
| | | | 2.841; 2.888 | | | |
| | | | | O | CH | 3.289 |
| | | | | phosphonate | aromatic | |
| | | | | Edge to face | Edge to face | 3.623 |
| | | | | Edge to edge | Edge to edge | 3.271 |

Conformations adopted by the chlorhexidine molecule in the co-crystal:

| | | C4diP-Chlorhexidine |
|---|---|---|
| | | co-crystal |
| Chlorhexidine | | Conformation |
| 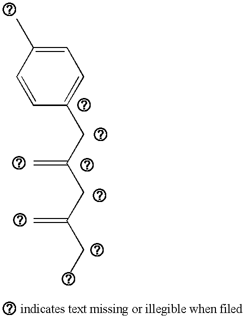 | 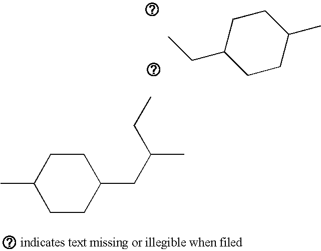 | C1 [symbol] N1 [symbol] C2 [symbol] N3 = −10.10 N1 [symbol] C2 [symbol] N3 [symbol] C3 = 150.35 C2 [symbol] N3 [symbol] C3 [symbol] N5 = 148.29 N3 [symbol] C3 [symbol] [symbol] [symbol] = −3.49 C3 [symbol] N5 [symbol] C4 [symbol] C5 = −165.38 N5 [symbol] C4 [symbol] C5 [symbol] C6 = 175.85 C4 [symbol] C5 [symbol] C6 [symbol] C7 = 178.79 C5 [symbol] C6 [symbol] C7 [symbol] C8 = −21.45 C6 [symbol] C7 [symbol] C8 [symbol] C9 = −170.27 C7 [symbol] C8 [symbol] C9 [symbol] N6 = 48.79 C8 [symbol] C9 [symbol] N6 [symbol] C10 = [symbol] C9 [symbol] N6 [symbol] C10 [symbol] N8 = −9.68 N6 [symbol] C10 [symbol] N8 [symbol] C11 = 146.70 C10 [symbol] N6 [symbol] C11 [symbol] N10 = 163.75 N8 [symbol] C11 [symbol] N10 [symbol] C12 = −2.00 |
| 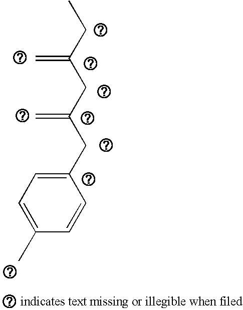 | | |

[symbol]

indicates data missing or illegible when filed

### Example 3

### chlorhexidine/dimethoxycarboxy calix[4]arene co-crystal

Dimethoxycarboxy calix[4]arene (25,27-bis(methoxycarboxy)-26,28-dihydroxycalix[4]arene) is represented by the following formula:

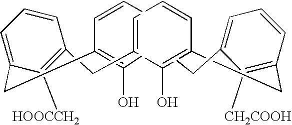

The chlorhexidine/dimethoxycarboxycalix[4]arene co-crystal is prepared according to the general procedure described above.

The crystallographic data are as follows:

| | Space | | | | | | | Molecular | | |
|---|---|---|---|---|---|---|---|---|---|---|
| Symmetry | group | A | B | C | α | β | γ | mass | Volume | R |
| triclinical | P-1 | 10.104 | 14.629 | 19.3 | 69.71 | 87.37 | 64.4270 | 960.23 | 2676.3 | 0.33 |
| | | (2) | (3) | 76 (4) | (3) | (6) | (10) | | (9) | |

The direct interactions between the dimethoxycarboxycalix[4]arene and chlorhexidine are as follows:

| | Hydrogen bond | | | Dipolar interactions | | |
|---|---|---|---|---|---|---|
| | calixarene | medication | distance [λ] | calixarene | medication | distance [λ] |
| Functions | O acid | N amine | 2.972 | Aromatic | CH₂ | 3.863; 3.824 |
| involved | | | | CH | | |
| | O acid | N imine | 2.916; 2.925; | CH₂ | CH₂ | |
| | | | 2.917; 2.867 | | | |
| | O phenolic | O amine | 2.981 | O phenolic | N amine | 3.222 |
| | | | | CH₂ | CH | 3.687 |
| | | | | | Aromatic | |

Conformations adopted by the chlorhexidine molecule in the co-crystal:

| | | C4diA-Chlorhexidine |
|---|---|---|
| | | co-crystal |
| Chlorhexidine | | Conformation |
| 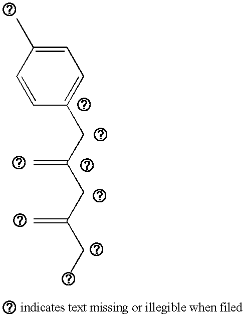 | 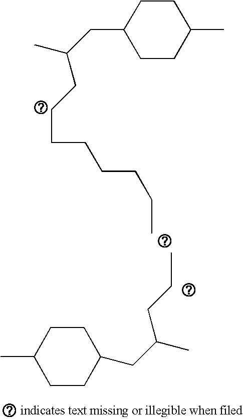 | C1 [symbol] N1 [symbol] C2 [symbol] N3 = −141.15 N1 [symbol] C2 [symbol] N3 [symbol] C3 = 161.79 C2 [symbol] N3 [symbol] C3 [symbol] N5 = 139.99 N3 [symbol] C3 [symbol] N5 [symbol] C4 = −6.15 C3 [symbol] N5 [symbol] C4 [symbol] C5 = 75.46 N5 [symbol] C4 [symbol] C5 [symbol] C6 = 51.68 C4 [symbol] C5 [symbol] C6 [symbol] C7 = 177.30 C5 [symbol] C6 [symbol] C7 [symbol] C8 = [symbol] C6 [symbol] C7 [symbol] C8 [symbol] C9 = [symbol] C7 [symbol] C8 [symbol] C9 [symbol] N6 = 63.07 C8 [symbol] C9 [symbol] N6 [symbol] C10 = −177.95 C9 [symbol] N6 [symbol] C10 [symbol] N8 = [symbol] N6 [symbol] C10 [symbol] N8 [symbol] C11 = −151.17 C10 [symbol] N8 [symbol] C11 [symbol] N10 = −159.9 N8 [symbol] C11 [symbol] [symbol] [symbol] C12 = 4.41 |
| 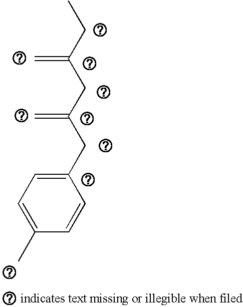 | | |

[symbol]

indicates data missing or illegible when filed

### Example 4

### Tetracaine/calix[4]arene parasulfonic acid co-crystal

The Tetracaine/calix[4]arene parasulfonic acid co-crystal is prepared according to the general procedure described above.

The crystallographic data are as follows:

| | Space | | | | | | | Molecular | | |
|---|---|---|---|---|---|---|---|---|---|---|
| Symmetry | group | A | B | C | α | β | γ | mass | Volume | R |
| triclinical | P-1 | 13.6140 | 13.8940 | 27.7950 | 97.6190 | 162.9336 | 98.489 | 1133.02 | 4891.5 | 0.09 |
| | | (3) | (4) | (4) | (3) | (10) | (2) | | (2) | |

The direct intra-actions between the calix[4]arene parasulfonic acid and the Tetracaine are as follows:

| | Hydrogen bond | | | Dipolar interactions | | |
|---|---|---|---|---|---|---|
| | calixarene | medication | distance [λ] | calixarene | medication | distance [λ] |
| Functions | sulfonate | amine | 2.806; 2.849 | CH | CH₂ | 3.894 |
| involved | sulfonate | ch₂ alkyl | 3.160 | sulfonate | CH₂ alkyl | 3.565 |
| | | | | sulfonate | CH₂ | 3.010; 3.210; |
| | | | | | | 3.302 |

Conformations adopted by the tetracaine molecule in the co-crystal:

| | C4S-Tetracain co-crystal | |
|---|---|---|
| Tetracaine | Conformations | |
| 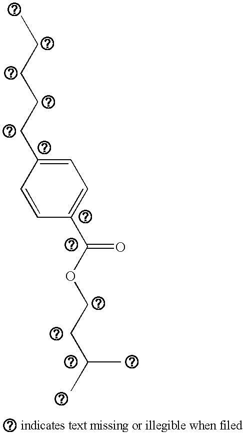 | 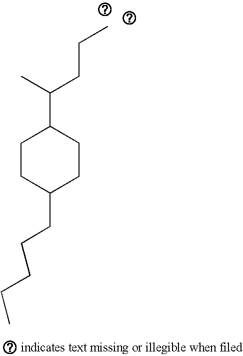 | C1 [symbol] N [symbol] C3 [symbol] C4 = −174.20 N [symbol] C3 [symbol] C4 [symbol] O1 = −56.10 C3 [symbol] C4 [symbol] O1 [symbol] C5 = −164.12 C4 [symbol] O1 [symbol] C6 = −178.62 C7 [symbol] C8 [symbol] C9 [symbol] C10 = 176.64 C8 [symbol] C9 [symbol] C10 [symbol] C11 = −179.01 C9 [symbol] C10 [symbol] C11 [symbol] C12 = 179.14 |
| | 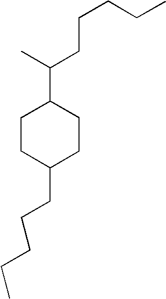 | C1 [symbol] N [symbol] C3 [symbol] C4 = −170.87 N [symbol] C3 [symbol] C4 [symbol] O1 = −172.10 C3 [symbol] C4 [symbol] O1 [symbol] C5 = −106.40 C4 [symbol] O1 [symbol] C6 = −170.58 C7 [symbol] C8 [symbol] C9 [symbol] C10 = −177.20 C8 [symbol] C9 [symbol] C10 [symbol] C11 = −172.72 C9 [symbol] C10 [symbol] C11 [symbol] C12 = 179.06 |
| | 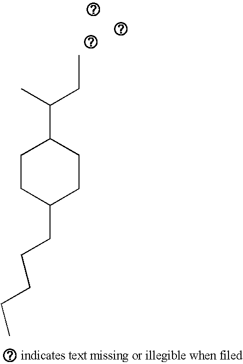 | C1 [symbol] N [symbol] C3 [symbol] C4 = [symbol] N [symbol] C3 [symbol] C4 [symbol] O1 = 174.94 C3 [symbol] C4 [symbol] O1 [symbol] C5 = [symbol] C4 [symbol] O1 [symbol] C6 = −176.16 C7 [symbol] C8 [symbol] C9 [symbol] C10 = 179.39 C8 [symbol] C9 [symbol] C10 [symbol] C11 = 178.48 C9 [symbol] C10 [symbol] C11 [symbol] C12 = 176.46 |
| | 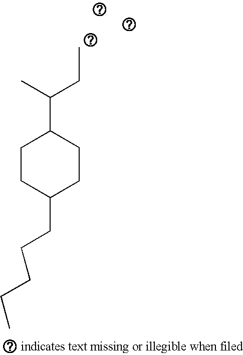 | C1 [symbol] N [symbol] C3 [symbol] C4 = −176.90 N [symbol] C3 [symbol] C4 [symbol] O1 = −67.71 C3 [symbol] C4 [symbol] O1 [symbol] C5 = −172.97 C4 [symbol] O1 [symbol] C6 = −176.93 C7 [symbol] C8 [symbol] C9 [symbol] C10 = 176.42 C8 [symbol] C9 [symbol] C10 [symbol] C11 = −178.65 C9 [symbol] C10 [symbol] C11 [symbol] C12 = −178.09 |

[symbol]

indicates data missing or illegible when filed

### Example 5

### Tamoxifen/calix[4]arene parasulfonic acid co-crystal

The Tamoxifen/calix[4]arene parasulfonic acid co-crystals are prepared according to the general procedure above.

The crystallographic data are as follows:

| | Space | | | | | | | Molecular | | |
|---|---|---|---|---|---|---|---|---|---|---|
| Symmetry | group | A | B | C | α | β | γ | mass | Volume | R |
| triclinical | P-1 | 13.8790 | 4.1200 | 31.9970 | 96.020 | 99.015 | 998.64 | 1713.24 | 6065.6 (2) | 0.08 |
| | | | | | | | (2) | | | |

The direct interactions between the calix[4]arene parasulfonic acid and the Tamoxifen are as follows:

| | Hydrogen bond | | | Dipolar interactions | | |
|---|---|---|---|---|---|---|
| | calixarene | medication | distance [A] | calixarene | medication | distance [A] |
| Functions | sulfonate | amine | 2.781; 2.766; | Sulfonate | CH₂ | 3.013 |
| involved | | | 2.784; 3.087 | | | |
| | sulfonate | CH₂alkyl | | OH | CH₂ | 3.039 |
| | | | | phenolic | | |
| | | | | sulfonate | N amine | 3.100 |
| | | | | sulfonate | Aromatic | 3.156 |
| | | | | | CH | |
| | | | | CH | CH₂ | 3.535; 3.548 |
| | | | | aromatic | | |

Conformations adopted by the Tamoxifen molecule in the co-crystal:

| | C4s-Tamoxifen co-crystal | |
|---|---|---|
| Tamoxifen | Conformations | |
| 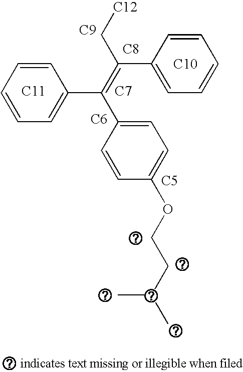 | 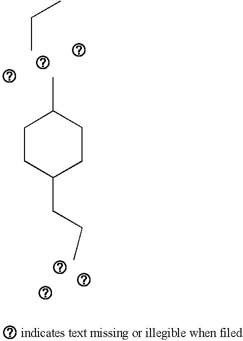 | C1 [symbol] N [symbol] C3 [symbol] C4 = 172.75 N [symbol] C3 [symbol] C4 [symbol] O = −59.22 C3 [symbol] C4 [symbol] O [symbol] C45 = 154.38 C6 [symbol] C7 [symbol] C8 [symbol] C9 = 175.81 C6 [symbol] C7 [symbol] C8 [symbol] C10 = −4.31 C10 [symbol] C8 [symbol] C7 [symbol] C11 = 177.20 C7 [symbol] C8 [symbol] C9 [symbol] C12 = 114.82 |
| | 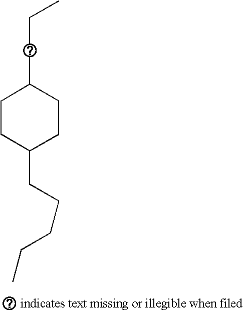 | C1 [symbol] N [symbol] C3 [symbol] C4 = 179.40 N [symbol] C3 [symbol] C4 [symbol] O = 53.64 C3 [symbol] C4 [symbol] O [symbol] C45 = 173.30 C6 [symbol] C7 [symbol] C8 [symbol] C9 = 177.05 C6 [symbol] C7 [symbol] C8 [symbol] C10 = 3.23 C10 [symbol] C8 [symbol] C7 [symbol] C11 = 179.03 C7 [symbol] C8 [symbol] C9 [symbol] C12 = 107.68 |
| | 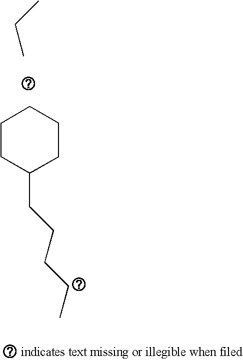 | C1 [symbol] N [symbol] C3 [symbol] C4 = 164.88 N [symbol] C3 [symbol] C4 [symbol] O [symbol] = −166.56 C3 [symbol] C4 [symbol] O [symbol] C45 = 175.28 C6 [symbol] C7 [symbol] C8 [symbol] C9 = 172.74 C6 [symbol] C7 [symbol] C8 [symbol] C10 = 4.9 C10 [symbol] C8 [symbol] C7 [symbol] C11 = 179.99 C7 [symbol] C8 [symbol] C9 [symbol] C12 = 113.57 |
| | 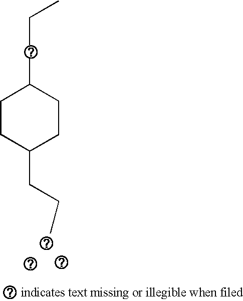 | C1 [symbol] N [symbol] C3 [symbol] C4 = 174.19 N [symbol] C3 [symbol] C4 [symbol] O = −55.78 C3 [symbol] C4 [symbol] O [symbol] C45 = [symbol] C6 [symbol] C7 [symbol] C8 [symbol] C9 = 178.36 C6 [symbol] C7 [symbol] C8 [symbol] C10 = −2.91 C10 [symbol] C8 [symbol] C7 [symbol] C11 = 178.57 C7 [symbol] C8 [symbol] C9 [symbol] C12 = 107.59 |

[symbol]

indicates data missing or illegible when filed

### Example 6

### Pilocarpine/calix[4]arene dihydroxyphosphonic acid co-crystal

The Pilocarpine/calix[4]arene dihydroxyphosphonic acid is prepared according to the general procedure described above.

The crystallographic data are as follows:

| | Space | | | | | | | Molecular | | |
|---|---|---|---|---|---|---|---|---|---|---|
| Symmetry | group | A | B | C | α | β | γ | mass | Volume | R |
| monoclinical | P21 | 15.5230 | 33.0520 | 18.830 | 90.00 | 309.433 | 90.00 | 1010.88 | 3607.86 | 0.08 |
| | | (4) | (4) | (5) | | (10) | | | (17) | |

The direct interactions between the calix[4]arene parasulfonic acid and the Pilocarpine are as follows:

| | Hydrogen bond | | | Dipolar interactions | | |
|---|---|---|---|---|---|---|
| | calixarene | medication | distance [A] | calixarene | medication | distance [A] |
| Functions | — | — | — | O phosphonate | Aromatic | 3.464 |
| involved | | | | | CH | |
| | | | | CH₂ | CH₂ | 3.870 |

Conformations adopted by the Pilocarpine molecule in the co-crystal:

| | C4diP-Pilocarpine co- |
|---|---|
| | crystal |
| Pilocarpine | Conformation |
| 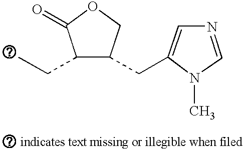 | 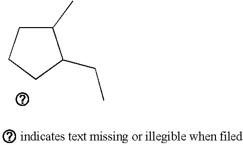 |

### Example 7

### Piribedil/calix[4]arene parasulfonic acid co-crystal

The Piribedil/calix[4]arene parasulfonic acid co-crystal is prepared according to the general procedure described above.

The crystallographic data are as follows:

| | Space | | | | | | | Molecular | | |
|---|---|---|---|---|---|---|---|---|---|---|
| Symmetry | group | A | B | C | α | β | γ | mass | Volume | R |
| monoclinical | P21/c | 12.5250 | 29.822 | 16.9630 | 90.00 | 90.00 | 90.00 | 1386.68 | 5472.9 | 0.09 |
| | | (4) | (9) | (8) | | | | | (3) | |

The direct interactions between the calix[4]arene parasulfonic acid and the Piribedil are as follows:

| | Hydrogen bond | | | Dipolar interactions | | |
|---|---|---|---|---|---|---|
| | calixarene | medication | distance [λ] | calixarene | medication | distance [A] |
| Functions | sulfonate | amine | 2.906; 2.738 | sulfonate | amine | 3.053 |
| involved | | | | sulfonate | CH | 2.797; 3.738 |
| | | | | | | 3.220 |
| | | | | CH | O (furan) | 3.157 |
| | | | | Edge-to-face | Edge-to-face | 3.02 |
| | | | | T-shape | T-shape | 3.70 |

Conformations adopted by the Piribedil molecule in the co-crystal:

| | C4S-Piribedil co-crystal |
|---|---|
| Piribedil | Conformation |
| 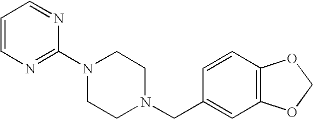 | 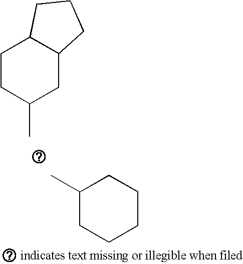 |

1. A co-crystal of at least one molecule of calix[n]arene or one of its derivatives, and at least one biologically active molecule.

2. A co-crystal according to claim 1, in which the said at least one calix[n]arene complies with the following formula (I): 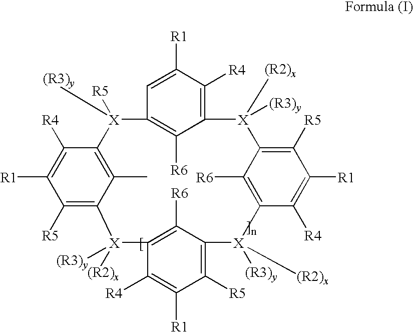
in which:
n is an integer number chosen from 1, 2, 3, 4, 5, 6, 7, 8, 9, 10, 11, 12, 13, 14, 15, 16, 17, 18, 19, and 20,
R1 represents a polar group, in particular chosen from the group comprising the hydroxyl, ether, carboxylic acid, sulfonic acid, phosphonic acid, sulfonamide, amide and ester functions, or an alkyl, alkene, alkyne, or acyl group, linear, branched, or cyclic, or an aryl, arylalkyl or alkylaryl group, or heterocyclic compound, possibly substituted, in particular by a polar group,
R2, R3, R4 and R5 each represent independently from one another a hydrogen atom, an alkyl, alkene or alkyne group, possible substituted, in particular by a polar group, in particular by a hydroxyl or ether function, in particular carrying an alkyl, alkene or alkyne group,
X represents an atom chosen from carbon, oxygen, sulfur and nitrogen,
x and y each represent independently of each other 0 or 1, and
R6 represents a polar group, in particular chosen from the group comprising the hydroxyl, ether, carboxylic acid, sulfonic acid, phosphonic acid, sulfonamide, amide and ester functions, or an alkyl group, branched or linear, possibly substituted by a polar group.

3. A co-crystal according to claim 2, in which the R4 and R5 groups are identical, and in particular represent a hydrogen atom.

4. A co-crystal according to claim 2, in which X is a carbon atom, and the carbon atoms carrying the R2 and R3 groups all have the same configuration, and in particular are all (S) or all (R).

5. A co-crystal according to claim 1, in which the said at least one calix[n]arene or the said at least one of its derivatives is chosen from the group comprising calix[4]arene (25,27-bis(dihydroxyphosphoriloxy)-26,28-dihydroxycalix[4]arene)dihydrophosphonic acid, calix[4]arene (5,11,17,23-tetrakis(p-sulfonic acid)-25,26,27,28-tetrahydroxycalix[4]arene) parasulfonic acid, dimethoxycarboxy calix[4]arene (25,27-bis(methoxycarboxy)-26,28 dihydroxycalix[4]arene, tetramethoxycarboxy calix[4]arene (25,26,27,28-tetra(methoxycarboxy)calix[4]arene, tetraproprioxycarboxy calix[4]arene (25,26,27,28-tetra(propioxycarboxy)calix[4]arene, calix[6]arene (5,11,17,23,29,35-hexasulfonic acid 37,38,39,40,41,42-hexahydroxycalix[6]arene parasulfonic acid, calix[8]arene (5,11,17,23,29,35,41,47-octasulfonic acid-50,51,52,53,54,56,57-octahydroxycalix[8]arene)parasulfonic acid, tetraacylcalix[4]arene(5,11,17,23-tetraacylcalix[4]arene), tetrahydroxycalix[4]arene), tetraacylcalix[6]arene(5,11,17, 23,29,35-tetracylcalix[6]arene).

6. A co-crystal according to claim 1, in which the said at least one biologically active molecule comprises at least one unit able to form at least one non-covalent interaction with at least one complementary unit of the said at least one calix[n]arene or of the said at least one of its derivatives.

7. A co-crystal according to claim 1, in which the said at least one biologically active molecule is a pharmaceutically active principle.

8. A co-crystal according to claim 1, in which the said at least one biologically active molecule is chosen from the group comprising N,N′-bis(4-chlorophenyl)-3,12-diimino-2,4,11,13-tetraazatetradecanediimidamide, 2-(dimethylamino)ethyl 4-(butylamino)benzoate, (Z)-2-[4-(1,2-diphenyl-1-butenyl)phenoxy]-N,N-dimethylethanamine, (3S-cis)-3-ethyldihydro-4-[(1-methyl-1H-imidaxol-5-yl)methyl]-2(3H)-furanone and 2-[4(1,3-Benzodioxol-5-ylmethyl)-1-piperazynyl]pyrimide.

9. A co-crystal according to claim 1, the said co-crystal having a solubility in water greater by at least 10% than that of the biologically active molecule in neutral form and/or in salt form.

10. A composition comprising at least one co-crystal, at least one calix[n]arene, or at least one of its derivatives, and at least one biologically active molecule in a pharmaceutically or physiologically acceptable carrier.

11. A medication comprising at least one co-crystal of at least one calix[n]arene, or at least one of its derivatives, and of at least one biologically active molecule.

12. Use of at least one co-crystal of at least one calix[n]arene, or at least one of its derivatives, and at least one biologically active molecule for preparing a medication, in particular intended for treating infectious diseases, in particular bacterial and/or viral, parasitic diseases, fungal infection diseases, prion diseases, allergic diseases, cardiovascular diseases, dermatological diseases, rare diseases (referred to as orphan diseases), genetic diseases, in particular classed by organ, apparatus or function or by region of the globe, chromosome diseases, in particular due to an abnormality in number or to a deletion, illnesses from intoxication, illnesses due to cell degenerescence, in particular cancers, leukaemias and Alzheimer's and Parkinson's diseases, environmental diseases, in particular obesity, alcoholism and hypertension, diseases caused by deficiencies, auto-immune and inflammatory diseases, diseases having an uncertain cause or multiple causes, diseases of the eye, and tissue and cell trauma.

13. Use of at least one calix[n]arene, or at least one of its derivatives, as an agent for forming a co-crystal with a biologically active molecule.

14. A method of preparing a co-crystal of at least one calix[n]arene or at least one of its derivatives, and at least one biologically active molecule comprising at least the steps consisting of:
putting at least one calix[n]arene, or one of its derivatives, together with at least one biologically active molecule, under conditions allowing the formation of co-crystals, and
recovering the said co-crystals.
